(19) United States
(12) Patent Application Publication
(10) Pub. No.: US 2004/0076618 A1
(43) Pub. Date: Apr. 22, 2004

(54) Placental preparation having antitumor activity

(75) Inventors: Shuang-Lin Lee, Taichung (TW); Shih-Lan Hsu, Taipei (TW)

Correspondence Address: Cohen, Pontani, Lieberman & Pavane, 551 Fifth Avenue, New York, NY, 10176, US

(73) Assignee: Ru-Chein YU

(21) Appl. No.: 10/405,543

(22) Filed: Apr. 2, 2003

(30) Foreign Application Priority Data
Oct. 18, 2002 (TW) 91124101

(51) Int. Cl. A61K 35/50

(52) U.S. Cl. 424/93.7; 424/583

(57) ABSTRACT

The present invention provides a placental preparation, its preparation method and medical uses in cancer treatments. Said placental preparation is a fraction having molecular weight less than 3 kDa obtained from a postpartum placenta of a mammal including human. The placental preparation according to the present invention has an excellent selective cytotoxicity toward tumor cells and in turn an excellent antitumor activity without any undesired side effect, and is thus very useful in cancer treatment.

### BACKGROUND OF THE INVENTION

1. Priority claim

Priority is claimed for this invention and application, corresponding application(s) having been filed in Taiwan on Oct. 18, 2002, No. 91124101.

The present invention relates to a placental preparation having antitumor activity, and to its preparation method and medical uses in cancer treatment.

2. Background of the Invention

Cancer represents the major cause of death in literally every country, and its incidence continues to rise worldwide. Accordingly medical researchers have been trying every effort to search for effective cancer treatment methods. The conventional treatment of cancer includes surgery, radiation and chemotherapy. Each of these therapies has serious side effects and other limitations in applications. The long-term results show a high rate of cancer recurrence after these treatments. However, cancer chemotherapy has progressed since its introduction into clinical practice and represents the most promising treatment modality. Various chemotherapy drugs, including doxorubicin, 5-fluorouracil, cisplatin, etoposide, taxol and gemeitabine, have been used to treat cancer. But all of these anticancer drugs affect not only pathological tumor cells, but also normal cells, especially bone marrow cells or intestinal epithelia with high turn over rate, which causes senous complications and toxicity. Therefore, it has prompted medical researchers to search for novel compounds with potent anticancer effect for cancer treatment.

Recently, many of cancer chemotherapies use alternative medicines, including herbal therapies. Animal and plant extracts have been used in traditional medicines from ancient times. A large number of the active ingredients in present day medicines have been obtained from such folk medicines as a result of modern science. In China, the history of animal extracts used as pharmaceutical preparations is extensive, and even at present, many animal products are incorporated into Chinese traditional medicines. Extracts from placenta is reported in many researches to have both cell activating characteristics and excellent ultraviolet absorption characteristics. For biological preparations based on placenta, there have been placental globulin, placental tissue fluid and placental tissue plasma.

In cancer treatment, since 1975 Russian physician, Dr. Valentin I. Govallo, has began treating cancer patients with an extract of chorionic villi from placentas obtained following live full-term deliveries (Govallo V I, Immunology of Pregnancy and Cancer; Nova Science Publishers, inc., 1993). This therapy is claimed to be different from chemotherapies and common immunotherapies that are cytotoxic, and is designed to weaken or suppress factors within the tumor that “turn off” the normal immune defense of the patient, and enable the patient's immune system to respond against the cancer and in turn control or eliminate the cancer. Therefore, this therapy inhibits or kills tumor cells through the patient's immune system.

U.S. Pat. No. 5,516,754 mentions a cancer treatment method using a combination of the photosynthesis system of plants and the embryonic tissue of mammals, which is useful for selective dissolution of cancer cells. The combined active ingredients are two protein fractions or protein mixtures derived from different sources, including a protein fraction with oxygen activity (liberation of oxygen from water under the influence of light and/or heat) derived from the photosynthetic system of plants and a protein mixture derived from calf or sheep embryos and fetuses. Said protein mixture comprises protein components showing a main band at 62±10 kDa and secondary bands at 43±10 LDa and 13.8±5 kDa. Said US patent stresses that the combination of active ingredients shows excellent selectivity in dissolution of tumor cells but no comparable effect is obtained when one of the two fractions is administered on its own.

CN 1235026A discloses a placenta peptide injection solution and preparation method thereof. Said placenta peptide product is an aqueous extract of placenta (1:1 v/w) having enhancement and adjustment effects on immune system and may be used as an “auxiliary medicine” in cancer treatment. This publication is silent about the selective cytotoxicity toward tumor cells or the antitumor effects of said placenta peptide product per se. Moreover, its preferred embodiment is directed to the extract fraction less than 10 kDa.

EP 1 133 990 A1 provides an antitumor composition consisting of a fraction of gravid uterus or mammalian placenta. The features and source of effect of said composition reside in the extract fraction having molecular weight lower than 10 kDa of gravid uterus or placenta of not human mammals taken during “the period of embryonal differentiation”. However, the biological experimental results show that the antitumor effect of said composition is not consistent. One of the experimental results shows the extract fraction having molecular weight lower than 10 kDa is significantly inferior to the uterus total extract in inhibition of tumor cell growth.

Accordingly, for the cancer treatment use of placental preparations, so far there has been no definite, reliable, effective preparation. The present inventors have conducted extensive and intensive studies in this regard and have found that a fraction having molecular weight less than 3 kDa obtained from a postpartum placenta of a mammal including human has a selective cytotoxicity toward tumor cells and in turn an excellent antitumor activity, and is thus very useful in cancer treatment, thereby completing the present invention.

3. Detailed Description of the Invention

The present inventors use fresh postpartum placentas from mammals such as, for example, porcine, cow, goat, rabbit and including human to obtain the fraction having molecular weight less than 3 kDa, which is then evaluated for cytotoxicity and antitumor activity by in vitro cell cultures and in vivo nude mice assay.

The placental preparation according to the present invention may be produced from fresh postpartum placentas from mammals such as, for example, porcine, cow, goat, rabbit and including human, preferably porcine placenta. The preparation process comprises rinsing and mincing the placenta, removing impurities, separating the fraction having molecular weight less than 3 kDa, and sterilization.

Removal of impurities such as tissue lumps may be carried out through centrifugation, typically at 2 to 8° C. under 10,000×g for 20 to 40 minutes, preferably at 4° C. for 30 minutes.

The placental preparation according to the present invention is the fraction having molecular weight less than 3 kDa, and hence any separation technique capable of separating a fraction having molecular weight less than 3 LDa can suitably be used in the present invention. For example, filtration with Centricon 3 kDa (Millipore Corporation, Mass. 01730 USA) may be employed to obtain the placental fraction having molecular weight less than 3 kDa. For sterilization, 0.22 μl m micropore sterilization filtration membrane may be used.

Trypan blue dye exclusion method which directly determines cell number using hemocytometer is employed in the present invention to effectively evaluating the cytotoxic effect. Also, an in vivo assay based on the xenograft nude mice model is used to evaluate the antitumor activity.

The placental preparation according to the present invention has selective cytotoxicity toward cancer tumor cell and antitumor activity without side effect, and is thus relatively safe and very useful in cancer treatment. The present invention therefore also relates to the medical uses of said placental preparation in cancer treatment. In practice, suitable pharmaceutical compositions such as, for example, injection and oral solutions, can be manufactured from the placental preparation according to the present invention for medical applications.

### BRIEF DESCRIPTION OF THE DRAWINGS

FIG. 1 shows the cytotoxic effects of the placental preparation according to the present invention on various human cancer cell lines.

FIG. 2 shows the inhibition of tumor growth in nude mice by the placental preparation according to the present invention.

FIG. 3 shows the influences of the placental preparation according to the present invention on survival rate of nude mice.

FIG. 4 shows the influences of the placental preparation according to the present invention on body weight of nude mice.

### EXAMPLE

Preparation:

Fresh postpartum placentas including those from porcine, cow, goat, rabbit and human were washed with distilled water, minced, and centrifuged at 10,000×g, at 2 to 8° C. for 20 to 40 minutes to remove precipitate of tissue lumps. Supernatants were centrifuged and filtered with Centricon 3 kDa. The filtered fraction (molecular weight less than 3 kDa) was then sterilized using 0.22 μm filter-tube, and stored at −80° C. before use.

Cytotoxicity and Antitumor Activity Assays

Cell Lines:

Human cancer cell lines were obtained from ATCC, and cultured according to ATCC procedures. That is, Hepatoma Hep3B and HepG2 cells were maintained in 10% fetal bovine serum (FBS)-DMEM; lung cancer H460 cells were cultured in 10% FBS-RPMI 1640 medium; melanoma A2058 cells were maintained in 10% FBS-DMEM. The cultured medium was changed every two days.

In Vitro Cytotoxicity Assay:

To determine the concentrations of placental preparation required for killing tumor cells in vitro, 1×10⁵ tumor cells/well were seeded into 12-well plates and incubated in the absence or presence of a series of dilutions of placental preparation in a humidified atmosphere of 5% CO₂ at 37° C. After treatment for 48 hours, cell viability in each well was measured using trypan blue dye exclusion method. The control wells in the absence of placental preparation were regarded as 100%, and the percentage of cell viability in each well was calculated.

In Vivo Nude Mice Assay:

Female BALB/c nude mice (about 6 to 8 weeks old) were maintained in a pathogen-free environment for one week. When 2×10⁶ cells were inoculated s.c. into right flank of nude mice, all mice formed a tumor within 14 days. Test groups were divided as follows: 1) buffer solution-treated control (shown as • in the figures); 2) treatment group where from 14 days after tumor inoculation, 0.5 ml of porcine placental preparation administrated i.p. daily for 12 weeks (shown as ▪ in the figures); 3) healthy control without inoculation (shown as ▴ in the figures). Each group consisted of 8 mice. The animals were investigated daily and tumor growth was determined by measurement of diameters of the tumor nodule in two dimensions with a caliper. Tumor size (cm3) was calculated by the use of the equation: A×B²/2, where A is longitudinal radius and B is transverse radius.

Statistical Analysis:

The significance of differences in tumor size and survival times among groups was analyzed using Student's t test.

Results:

Cytotoxic Effects of the Placental Preparation on Human Cancer Cell Lines

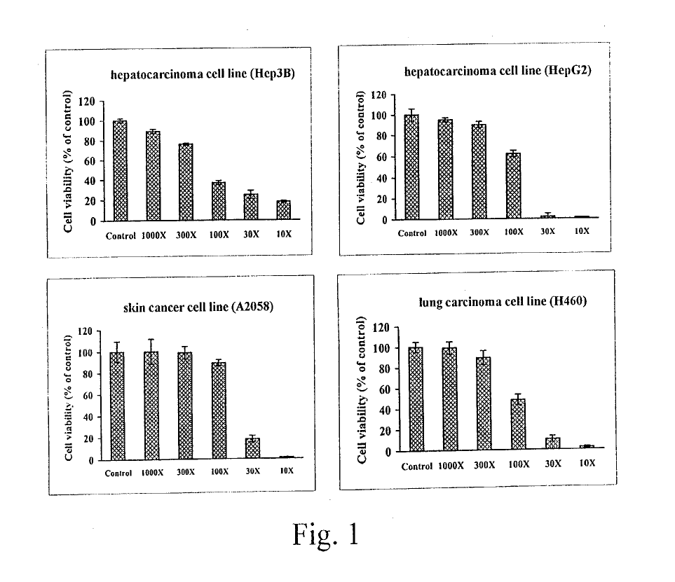

To evaluate the cytotoxic effect of the placental preparation according to the present invention on human hepatoma, lung carcinoma and melanoma cancer cells in vitro, Hep3B, HepG2, H460 and A2058 cells were plated at a density of 1×10⁵ cells/well in 12-well plates in the absence or presence of a series of diluted placental extract (1000×, 300×, 100×, 30× and 10× dilutions) in the medium, cells were harvested 48 hours after treatment. The number of viable cells of each treatment was directly determined by trypan blue dye exclusion method. As shown in FIG. 1, cells treated with the placental preparation according to the present invention exhibited a profound concentration-dependent reduction in their viability over the 48-hour test period. At 30× to 10× dilution, the placental preparation induced a strong cytotoxic effect in all tested cancer cells.

Effects of the Placental Preparation on Tumor Growth and Survival of Nude Mice

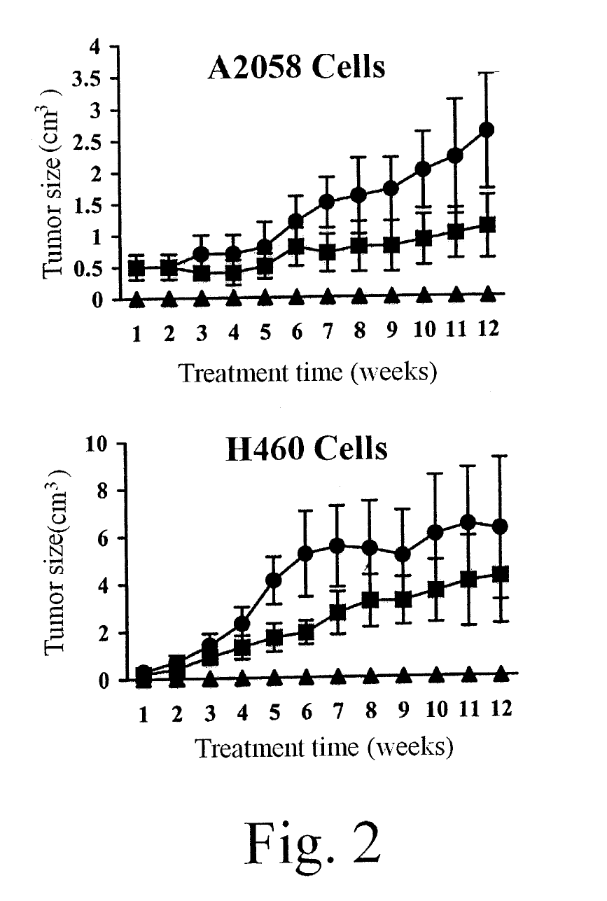

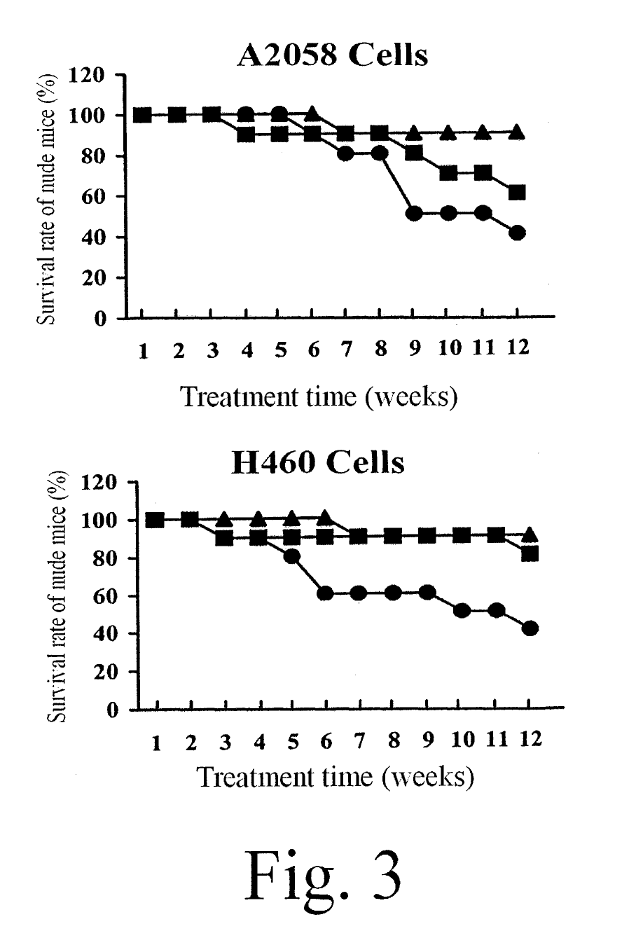

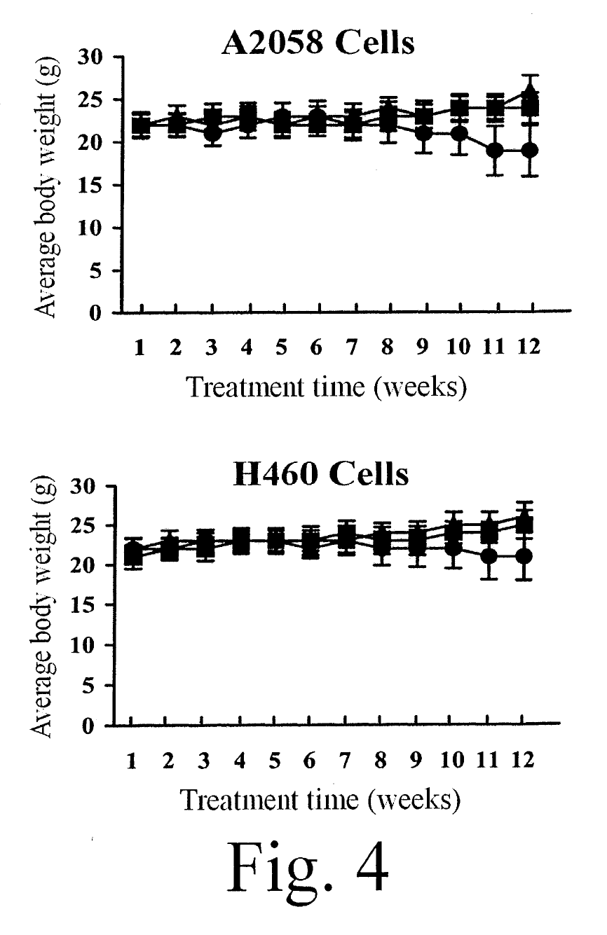

To evaluate the in vivo effects of the placental preparation according to the present invention on the tumor growth and survival of the tumor-bearing mice, the xenograft experiment using nude mice was performed. Daily i.p. administration of 0.5 ml the placental preparation showed a marked inhibitory effect on the lung (H460) and melanoma (A2058) tumor growth (FIG. 2). Moreover, treatment with the placental preparation resulted in a significant prolongation of the survival time, compared to control group (FIG. 3). Furthermore, daily i.p. treatment with 0.5 ml the placental preparation had an inhibitory effect on the tumor growth without any adverse side effect such as weight loss (FIG. 4), consequently resulting in a significant increase of life span.

It was confirmed from the above assays that the placental preparation with molecular weight less than 3 kDa according to the present invention significantly inhibited proliferation in several human tumor cell lines, including hepatoma Hep3B and HepG2 cell lines, lung cancer H460 cell line, and melanoma A2058 cell line. The in vivo nude mice assay demonstrated that daily i.p. administration of the placental preparation according to the present invention significantly inhibited the tumor growth and markedly improved the survival rate of tumor-bearing nude mice without any adverse side effect. These results indicated that the placental preparation having molecular weight less than 3 kDa according to the present invention has a potent antitumor effect, and is relatively safe without adverse side effects.

We I claim:

1. A placental preparation which is a fraction having molecular weight less than 3 kDa obtained from a mammalian placenta.

2. The placenta preparation according to claim 1, wherein said placenta is from a mammal selected form the group consisting of porcine, cow, goat, rabbit and human.

3. The placenta preparation according to claim 1, wherein said placenta is from porcine.

4. The placenta preparation according to claim 1 which is further concentrated or diluted for use.

5. A method for preparing the placental preparation as defined in claim 1, comprising rinsing the placenta with water, mincing it, removing precipitates, separating the fraction having molecular weight less than 3 kDa, and sterilization.

6. The method according to claim 5 further comprising a treatment step of concentration and dilution.

7. The method according to claim 5, wherein precipitates are removed by centrifugation.

8. The method according to claim 5, wherein sterilization is carried out with 0.22 μm micropore sterilization filtration membrane.

9. A pharmaceutical composition for cancer treatment comprising the placental preparation as defined in claim 1 and optional formulation aids.
